(19) United States
(12) Patent Application Publication
(10) Pub. No.: US 2015/0272497 A1
(43) Pub. Date: Oct. 1, 2015

(54) METHODS, SYSTEMS, AND COMPUTER READABLE MEDIA FOR VERIFYING THE ACCURACY OF MEDICAL TREATMENT IN ACCORDANCE WITH A TREATMENT PLAN

(71) Applicants: Nathan CHILDRESS, Bellaire, TX (US); Eli STEVENS, San Jose, CA (US); David EKLUND, San Francisco, CA (US)

(72) Inventors: Nathan CHILDRESS, Bellaire, TX (US); Eli STEVENS, San Jose, CA (US); David EKLUND, San Francisco, CA (US)

(73) Assignee: MOBIUS MEDICAL SYSTEMS, LP, Bellaire, TX (US)

(21) Appl. No.: 14/224,101

(22) Filed: Mar. 25, 2014

(51) Int. Cl. A61B 5/00; A61B 19/00; G06F 19/00

(52) U.S. Cl. CPC A61B 5/4848; G06F 19/3406; A61B 5/7246; A61B 19/50; A61B 2560/0475

(57) ABSTRACT

Applicants have created systems, methods, and computer readable media for verifying the accuracy of medical treatment in accordance with a treatment plan. The method can include the step of receiving medical treatment data including one or more treatment fields and the step of comparing a sample of a segment of treatment plan data with a sample of a first treatment field. The method can further include the step of comparing the segment with the first treatment field if the compared samples match within a first tolerance and the step of generating output data including the results of the step of comparing the segment with the first treatment field if a match occurs within the first tolerance. Through the inventions described herein, a software-based quality assurance analysis can be performed to quickly and accurately verify a patient's dosage in a variety of medical treatments including intensity-modulated radiation therapy.

### CROSS REFERENCE TO RELATED APPLICATIONS

Not applicable.

### STATEMENT REGARDING FEDERALLY SPONSORED RESEARCH OR DEVELOPMENT

Not applicable.

### REFERENCE TO APPENDIX

Not applicable.

### BACKGROUND OF THE INVENTION

1. Field of the Invention

The inventions disclosed and taught herein relate generally to systems, methods, and computer readable media for verifying the accuracy of medical treatment. In one of the aspects, the invention specifically relates to systems, methods, and computer readable media for comparing and analyzing a patient's treatment plan with the particular dosages and treatments performed on the patient to assess the level of quality assurance of a particular medical treatment. In further aspects, the inventions relate to systems, methods, and computer readable media to perform quality assurance analyses to verify a patient dosage quickly and accurately in a variety of medical treatments including intensity-modulated radiation therapy.

2. Description of the Related Art

The inventions disclosed and taught herein are directed to improved systems, methods, and computer readable media for verifying the accuracy of medical treatment. Although these inventions can be used in numerous applications, the inventions will be disclosed in only a few of many applications for illustrative purposes.

Intensity-Modulated Radiation Therapy (IMRT) is a form of radiation therapy that utilizes radiation (e.g., ionizing radiation, radiation to eradicate malignant cells such as those associated with cancer, etc.). Typically, equipment, such as a linear particle accelerator, is employed to focus a beam of high-energy particles on a patient's tissue at certain discrete locations of the body containing malignant and/or cancerous cells. The beam can be reshaped, moved, and adjusted throughout the therapy in order to target only those cells that are deemed malignant, while leaving healthy cells unaffected by the treatment.

Because it is of critical import to minimize damage to healthy cells through unintended exposure to the particular accelerator's beam, doctors and other medical professionals and patients seek to ensure that the treatments are being properly applied. In order to verify the accuracy of the dose applied during a given treatment, quality assurance procedures must be implemented. Among other things, these quality assurance procedures serve to ensure that certain tissue was properly targeted, while the exposure of other, healthy tissue to the energy of the particular beam was minimized, or avoided altogether.

In the past, hardware-based systems have been implemented with the goal of verifying the quality of a patient's treatment. For example, FIG. 1 illustrates a flow diagram depicting a hardware-based prior art method for analyzing medical treatment information. In this example, the method 100 includes the step 102 of preparing an IMRT Quality Assurance (QA) plan. Once the plan is complete, the step 104 of setting up a measuring device is performed. The device must be first properly calibrated through the step 106 of calibration before the step 108 of delivering the QA fraction can occur.

After the fraction is delivered, the step 110 of importing the measured and Treatment Plan Verification (TPS) planar dose occurs. Then, the operator must perform step 112 of projecting onto the patient's anatomy. This step alone can take upwards of over fifteen minutes to complete. Finally, comparisons can be performed at step 114 and the results can be analyzed at step 116.

The drawbacks to this hardware-based systems are numerous. For example, being hardware-based, the entire complete process is time consuming and costly. Moreover, often additional steps are required to the complete the hardware-based process. For example, the use of a phantom (such as a piece of material, plastic, or the like with one or more embedded detectors) needs to be employed for the verification. Additionally, some prior art systems are limited to two dimensions such that the dose verification is limited to planar area with no reference to depth. This is problematic because the verification cannot be accurately determined in light of this constraint. Further, these two-dimensional systems are often limited to detectors disposed on a particular two-dimensional plane and, thus, are limited in this respect. Finally, these hardware-based system typically provide a lower resolution array, require a manual importation of the TPS dose, and cannot segregate among error sources.

What is required, therefore, are systems, methods, and computer readable media that are capable—among other things—of quickly and accurately performing quality assurance analysis and verification of dosage applied to a patient during a medical treatment that require less time, are less expensive, and provide a high resolution in three-dimensional space. Moreover, what is further required are systems, methods, and computer readable media that do not require ion-chamber, diode-array, EPID, film, or external measurements to determine a 3D dose delivered to a patient. Accordingly, the inventions disclosed and taught herein are directed to systems, methods, and computer readable media that overcome the problems as set forth above.

### BRIEF SUMMARY OF THE INVENTION

Applicants have created systems, methods, and computer readable media for verifying the accuracy of medical treatment in accordance with a treatment plan. The method can include the step of receiving medical treatment data including one or more treatment fields and the step of comparing a sample of a segment of treatment plan data with a sample of a first treatment field. The method can further include the step of comparing the segment with the first treatment field if the compared samples match within a first tolerance and the step of generating output data including the results of the step of comparing the segment with the first treatment field if a match occurs within the first tolerance. Through the inventions described herein, a software-based quality assurance analysis can be performed to quickly and accurately verify a patient's dosage in a variety of medical treatments including intensity-modulated radiation therapy.

The method can include the step of receiving medical treatment data including one or more treatment fields and the step of comparing a sample of a segment of treatment plan data with a sample of a first treatment field. Further, the method can include the step of comparing the segment with the first treatment field if the compared samples match within a first tolerance and the step of generating output data including the results of the step of comparing the segment with the first treatment field if a match occurs within the first tolerance.

Additionally, the method can include the step of comparing the sample of the segment with a sample of a subsequent treatment field if the comparison of the segment with the first treatment field did not result in a match within the first tolerance and the step comparing the segment with the subsequent treatment field if the compared samples match within a first tolerance. If a match occurs within the first tolerance, the method can further include the step generating output data including the results of the step of comparing the sample of the segment and the sample of a subsequent treatment field. Further, the method can include the step of analyzing the output data to assess the level of quality assurance of the medical treatment.

Finally, the method can further include the step of comparing the sample of the segment with a sample of another subsequent treatment field if the comparison of the samples did not match within the first tolerance and the step of comparing a sample of a subsequent segment of treatment plan data with a sample of a subsequent treatment field segment after completing the step of generating output data.

The computer readable medium can be configured to store a program that is adapted to execute instructions for performing a series of steps. The computer readable medium's program can perform the step of receiving medical treatment data including one or more treatment fields and the step of comparing a sample of a segment of treatment plan data with a sample of a first treatment field. Further, the computer readable medium's program can perform the step of comparing the segment with the first treatment field if the compared samples match within a first tolerance and the step of generating output data including the results of the step of comparing the segment with the first treatment field if a match occurs within the first tolerance.

Additionally, the computer readable medium's program can perform the step of comparing the sample of the segment with a sample of a subsequent treatment field if the comparison of the segment with the first treatment field did not result in a match within the first tolerance and the step comparing the segment with the subsequent treatment field if the compared samples match within a first tolerance. If a match occurs within the first tolerance, the computer readable medium can further perform the step generating output data including the results of the step of comparing the sample of the segment and the sample of a subsequent treatment field. Further, the computer readable medium's program can perform the step of analyzing the output data to assess the level of quality assurance of the medical treatment.

Finally, the computer readable medium can further perform the step of comparing the sample of the segment with a sample of a subsequent treatment field if the comparison of the segment with the first treatment field did not result in a match within the first tolerance and the step of comparing a sample of a subsequent segment of treatment plan data with a sample of a subsequent treatment field segment after completing the step of generating output data.

The system can include a computer readable medium configured to store a program that is adapted to execute instructions for performing a series of steps. The computer readable medium's program can perform the step of receiving medical treatment data associated with a first patient, wherein the medical treatment data includes a plurality of treatment fields and the step of comparing a first portion of a current segment of treatment plan data with a first portion of a current treatment field of the medical treatment data.

Additionally, the computer readable medium's program can perform the step of comparing the current segment with the current treatment field if the first portion of the current segment matches the first portion of the current treatment field within a first tolerance and the step of generating output data comprising the results of the comparing the current segment with the current treatment field step in response to the step of comparing the current segment. Finally, the computer readable medium's program can perform the step of repeating the comparing and generating steps described above.

The computer readable medium can further perform the step of comparing the first portion of the current segment to a first portion of a subsequent treatment field if the step of comparing the current segment does not result in a match within the first tolerance. Moreover, the step of setting the subsequent treatment field as the current treatment field and repeating the step of comparing the current segment with the current treatment field and the step of generating output data. Additionally, the computer readable medium's program can perform the step of setting a subsequent segment as the current segment and setting a subsequent treatment field as the current treatment field in response to the step of generating output data and the step of setting a flag associated with the current treatment field in response to the step of generating output data.

### BRIEF DESCRIPTION OF THE SEVERAL VIEWS OF THE DRAWINGS

The following figures form part of the present specification and are included to further demonstrate certain aspects of the present invention. The invention may be better understood by reference to one or more of these figures in combination with the detailed description of specific embodiments presented herein.

FIG. 1 illustrates a flow diagram depicting a hardware-based prior art method for analyzing medical treatment information.

FIG. 2 illustrates a first embodiment of medical data in accordance with the present invention.

FIG. 3 illustrates a flow diagram depicting a first embodiment of exemplary steps for carrying out a method for verifying the accuracy of medical treatment.

FIG. 4 illustrates a flow diagram depicting a second embodiment of exemplary steps for carrying out a method for verifying the accuracy of medical treatment.

FIG. 5A illustrates a second embodiment of medical data in accordance with the present invention.

FIG. 5B illustrates the second embodiment of medical data as depicted in FIG. 5A illustrating certain features in accordance with the present invention.

FIG. 5C illustrates the second embodiment of medical data as depicted in FIG. 5A illustrating additional features in accordance with the present invention.

FIG. 5D illustrates a second embodiment of exemplary steps for verifying the accuracy of medical treatment in accordance with the second embodiment of medical data as depicted in FIG. 5A.

FIG. 6 illustrates an embodiment of a computer readable medium configured to store an application for verifying the accuracy of medical treatment in accordance with certain aspects of the inventions described herein.

FIG. 7 illustrates an embodiment of a system for verifying the accuracy of medical treatment in accordance with certain aspects of the inventions described herein.

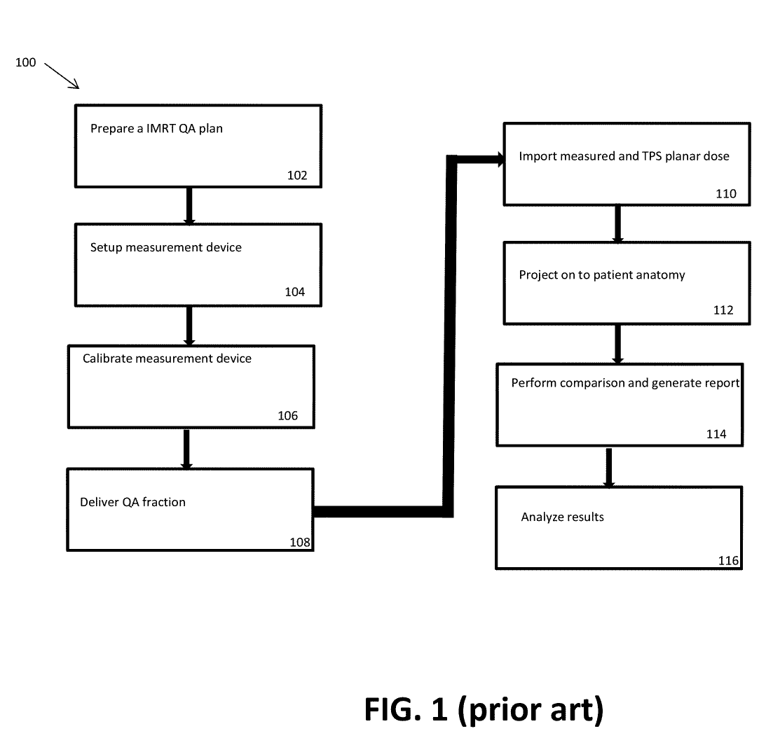

While the inventions disclosed herein are susceptible to various modifications and alternative forms, only a few specific embodiments have been shown by way of example in the drawings and are described in detail below. The figures and detailed descriptions of these specific embodiments are not intended to limit the breadth or scope of the inventive concepts or the appended claims in any manner. Rather, the figures and detailed written descriptions are provided to illustrate the inventive concepts to a person of ordinary skill in the art and to enable such person to make and use the inventive concepts.

### DETAILED DESCRIPTION

Applicants have created systems, methods, and computer readable media for verifying the accuracy of medical treatment in accordance with a treatment plan. The method can include the step of receiving medical treatment data including one or more treatment fields and the step of comparing a sample of a segment of treatment plan data with a sample of a first treatment field. The method can further include the step of comparing the segment with the first treatment field if the compared samples match within a first tolerance and the step of generating output data including the results of the step of comparing the segment with the first treatment field if a match occurs within the first tolerance. Through the inventions described herein, a software-based quality assurance analysis can be performed to quickly and accurately verify a patient's dosage in a variety of medical treatments including intensity-modulated radiation therapy.

For example, without reference to any specific figure, the inventions described herein can be employed to verify that a given linear particle accelerator (i.e., linac) accurately delivered a treatment dose to a patient in accordance with a doctor's and/or other medical professional's plan. In an exemplary and non-limiting illustrative embodiment, the inventions described herein can perform 3D verifications including gamma passing rate, verification of dose-volume histogram (DVH) objectives, etc. Further, the inventions described herein can deliver detailed quality assurance (QA) results for individual linac components including radiation jaws (i.e., course adjustments for shaping the linac beam into a rectangular or other geometrically-shaped beams), multi leaf collimators (MLC) (i.e., fine adjustments of the linac beam) on a leaf-by-leaf basis, and gantry (i.e., the linac component that rotates about the patient during his treatment), for example. With the inventions described herein, one can additionally verify that the treatment plan—as prescribed by a doctor or other medical professional—was successfully transferred to the appropriate Records and Verification (R&V) systems without error.

With the inventions described herein, error sources can be segregated (e.g., as calculation- or delivery-based) and reports can be more quickly and efficiently generated (e.g., in pdf or other formats) even while navigating complex Volumetric Arc Modulated Therapy (VMAT) plans. The analysis and verification performed in accordance with the inventions described herein can be performed quickly (e.g., in 1-5 minutes), in high resolution (e.g., MLC log data at approximately 0.1 mm) with the automatic importation of the TPS dose. Doctors and patients alike can, therefore, obtain an improved level of confidence knowing that a correct dose is being delivered to the patient the first fraction and every fraction afterwards when employing the inventions described herein.

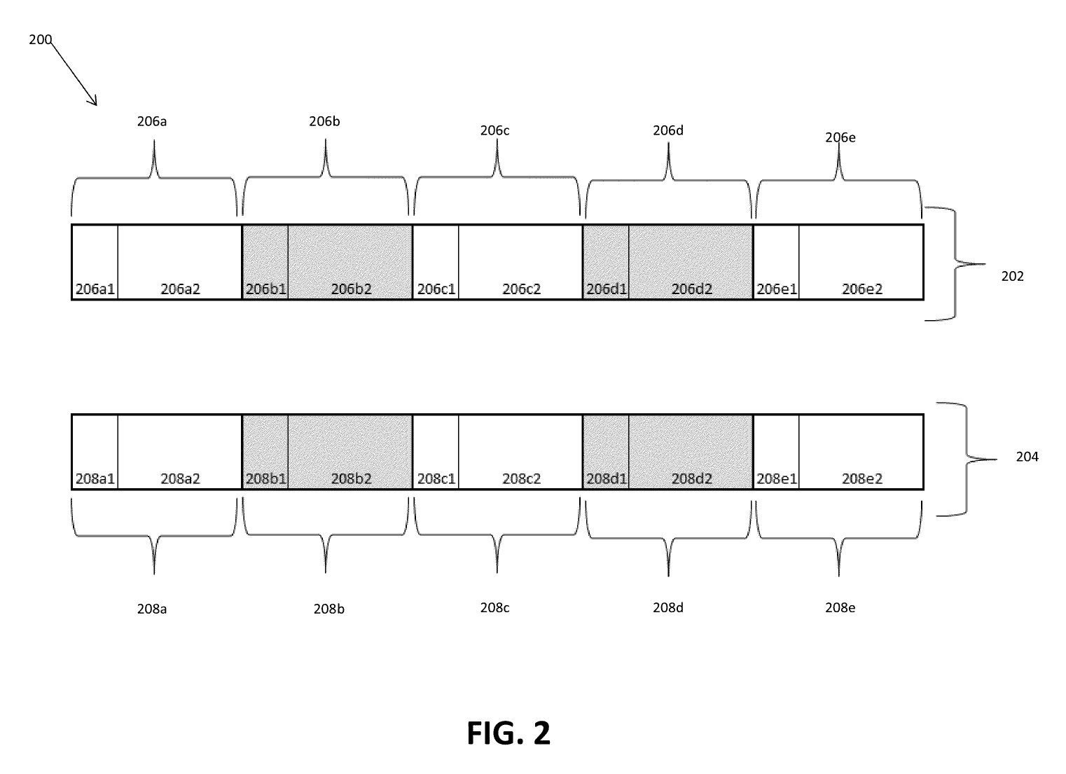

Turning now to the figures, FIG. 2 illustrates a first embodiment of medical data in accordance with the present invention. The medical data can include a data structure 200 that can include one or more data structures (e.g., linked list, tables, log files, or the like) or, alternatively, it can include raw data. As illustrated in FIG. 2, data structure 200 can include two separate linked lists embodied as log files. In an exemplary and non-limiting illustrative embodiment, these logs (e.g., the medical treatment data 202 as described in greater detail below) can include linac treatment logs (e.g., Varian DynaLogs, Varian Trajectory Logs, Elekta Mobius Logs, etc.) in order to calculate and verify the delivered 3D dose in a patient. Alternatively, other logs and/or formats of data are contemplated as well.

Although shown to be stored in contiguous memory, in the alternative, these data can be stored at various locations across one or more storage media (e.g., referenced by memory pointers or the like, or other memory addresses schemes, such as, for example, virtual memory techniques). The data structure 200 can include medical treatment data 202 and treatment plan data 204. The medical treatment data 202 can be supplied from the equipment performing the treatment (e.g., linac), or alternatively, provided from a separate system, computer, server, or the like. In other words, the medical treatment data 202 can include the actual data collected from the medical equipment that has performed the treatment (e.g., radiation therapy) on the patient. The equipment performing the treatment can include a linear accelerator or the like, or other equipment for performing radiation-type treatment and/or other medical treatment on a patient.

The medical treatment data 202 can include one or more treatment fields 206a-206e. Although five of such treatment fields are illustrated by this embodiment, more or fewer fields are contemplated as well. Additionally, although not shown in the figure, other data can be stored as part of the medical treatment data 202 or, in the alternative, stored in conjunction to, and associated with, the medical treatment data 202. For example, the patient's information (e.g., name, address, social security number, Universal Identification Number, etc.), date and time of medical treatment, doctor's names, treating facility name, etc. can be part of these medical treatment data 202 as well.

Each treatment field 206a-206e of medical treatment data 202 can include a first portion/first sample (e.g., 206a1, 206b, etc.) and a second portion/second sample (e.g., 206a2, 206b2, etc.). In other examples, these treatment fields 206a-206e can include more than two portions/samples. In one example, first portion (e.g., 206a1) contains a smaller amount of data than its treatment field's 206 respective second portion (e.g., 206a1). For example, in treatment field 206 contains a total of thirty steps, (these “steps” are described in greater detail below), then first portion/sample 206a1 can include, for example, ten of those thirty steps while second portion/sample 206a2 can include the remaining twenty. Although not specifically described, a greater or small ratio a first-to-second portions/samples are contemplated as well.

The “steps” contained within each treatment field 206a-206e can represent a series of positions and/or actions (e.g., rotations, movements between and among coordinates, etc.) to be performed for the treatment of a patient. For example, a field (e.g., 206a) can include a set of steps to instruct the equipment to direct energy, such as a linear accelerator beam, through a series of motions based on three-dimensional coordinates, rotational angles, and the like. These steps can be recorded as precise measurements of the device components including fractional monitor units (MU), MLC positions, jaw positions, gantry angles, collimator angles, couch angles, entry angles, beam energy, wedge insertion, etc. These positions/measurements can be recorded multiple times per second (e.g., 20-100/second, but either more or less refined sampling can be employed as well) and the tolerance levels (as described in greater detail below) can be set individually for the various angles, distances, etc. set forth in during the treatment.

To further illustrate, a treatment field 206a (for example) can instruct the equipment to direct a beam at a portion of a patient to perform medical treatment. During a patient's therapy treatment, the equipment can execute one or more of these actions within a discrete fields by turning the beam on and off at particular locations and throughout a series of rotations to execute the particular treatment required in accordance with a patient's treatment plan. A group of these steps, therefore, can comprise a particular field among the fields in the medical treatment data 202.

Because the medical treatment data 202 (i.e., data that includes steps actually performed on a patient during her treatment) often unintentionally diverges from the treatment actually prescribed by a doctor or other medical professional, the media treatment data 202 must be compared with the plan set forth by the patient's doctor. These data can be stored as the treatment plan data 204. As such, treatment plan data 204 can include the data that sets forth the steps that are intended to be performed on the patient. Once performed, a comparison can take place to determine the effectiveness of the dose during the patient's therapy and these data can be analyzed in accordance with this disclose to perform quality assurance analysis on the patient's treatment.

As described in conjunction with the medical treatment data 202, the treatment plan data can be broken into small discrete unit (e.g., segments). The treatment plan can include treatment plan data 204 that includes the plan selected by a doctor or other medical professional for each of the fields to be performed on the patient. In other words, just as the equipment can perform a series of discrete fields on a patient, the treatment plan data can include a plurality of discrete fields (referred to throughout as segments) describing the instructions intended to be performed on the patient in order to perform the proper treatment pursuant to the doctor or medical professional's recommendations. As discussed in greater detail below, the treatment plan data 204 are compared with the medical treatment data 202 in order to verifying the accuracy of the medical treatment. Such a comparison can be performed, for example, on a field-to-segment basis as described in greater detail below.

Each segment 208a-208e of treatment plan data 204 can include a first portion/first sample (e.g., 208a1, 208b, etc.) and a second portion/second sample (e.g., 208a2, 208b2, etc.). In other examples, these segments 208a-208e can include more than two portions/samples. In one example, first portion (e.g., 208a1) can contain a smaller amount of data than the segment's 208 second portion. Keeping with the example of thirty steps above, first portion/sample 208a1 can include, for example, ten of those thirty steps while second portion/sample 208a2 can include the remaining twenty. Although not specifically described, a greater or small ratio a first-to-second portions/samples are contemplated as well.

Each segment (e.g., 208a-208e) can represent one or more steps that are intended to be performed such that a match will occur between the segment and a corresponding treatment field. For example, as shown in FIG. 2, the treatment plan data 204 can include five segments 208a-208e. Once the treatment is performed, treatment fields 206a-206e can be recorded and stored. A comparison can then take place on a field-to-segment basis to determine how closely the treatment (e.g., field 206a) matched the plan (e.g., segment 208a).

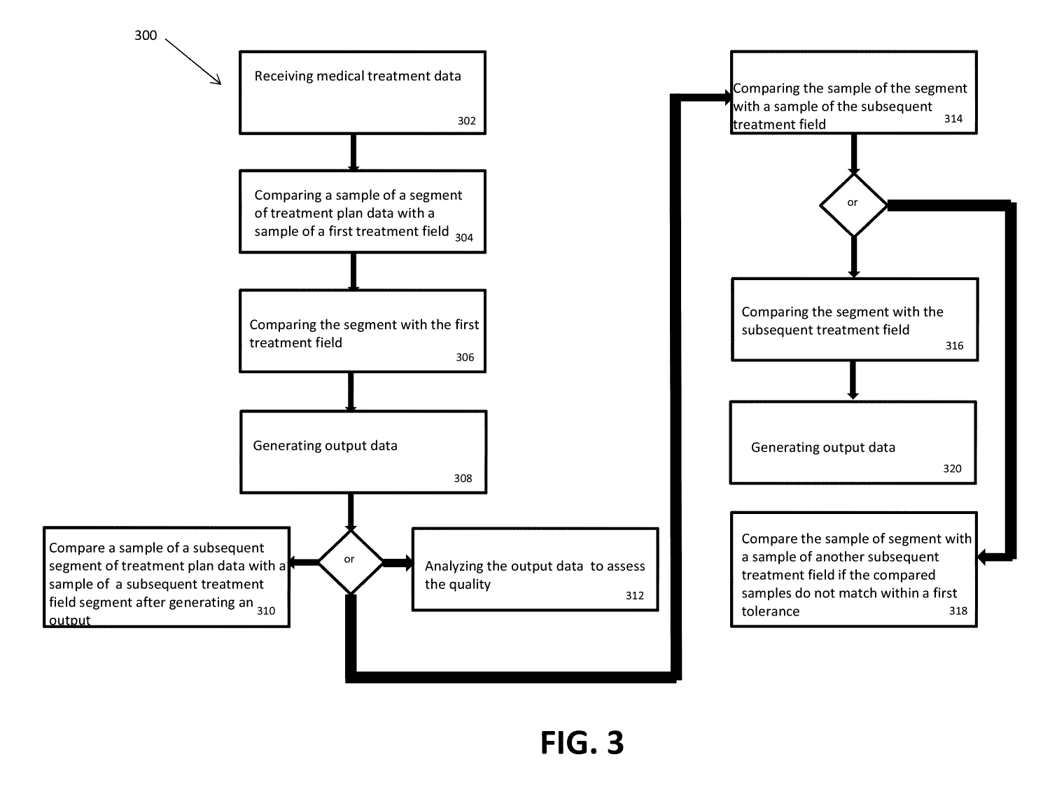

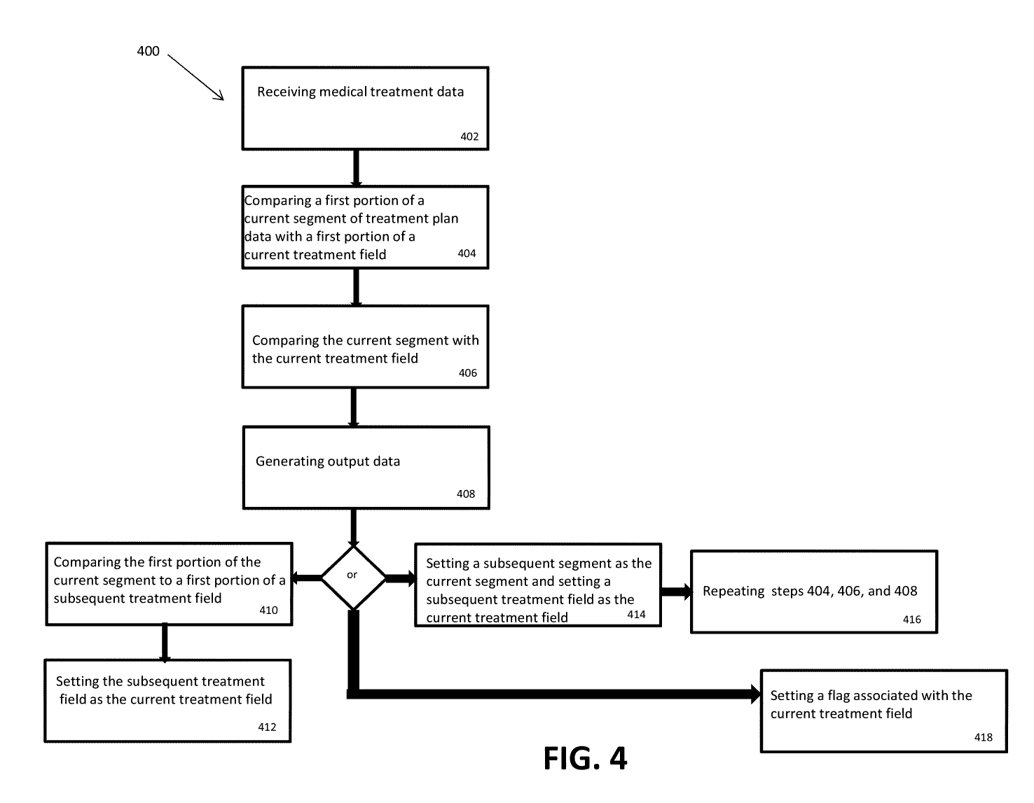

Because the treatment fields 206a-206e often are performed on the patient and/or stored in memory in a different order than they are stored in the treatment plan data 204, a simple comparison of each segment to treatment field (e.g., compare segment 208a to treatment fields 206a) could result in erroneous comparisons. Additionally, often it is difficult to determine whether or not the correct patient is even being compared with the treatment plan data because it is not uncommon for even the Universal Identification Number to change over the course of various treatments. As such, when comparing a given segment to a given treatment field, a determination must be made (within certain tolerances) whether or not the correct segment and field are being matched before any meaningful quality assurance analysis can take place. Given the context of the example described in conjunction with the data structure 200 of FIG. 2, FIGS. 3 and 4 illustrate exemplary embodiments of performing these comparisons in order to perform the necessary quality assurance analysis of a given patient's treatment dose.

FIG. 3 illustrates a flow diagram depicting a first embodiment of exemplary steps for carrying out a method for verifying the accuracy of medical treatment. The method 300 can include the step 302 of receiving medical treatment data including one or more treatment fields and the step 304 of comparing a sample of a segment of treatment plan data with a sample of a first treatment field. The step 304 of comparing a sample of a segment of treatment plan data with a sample of a first treatment field can be performed by comparing a portion of the first few instructions in a given segment with first few instructions of a given treatment field. In an exemplary and non-limiting illustrative embodiment, a field may include twenty discrete sets of coordinates and/or angles for which the equipment must navigate to compete a particular fields. In one example, the step 304 can include comparing a sample of the treatment field and segment of the treatment plan, such as the first five of these twenty steps.

Although this example employs twenty total steps, the first five of which are being compared, other examples are contemplated as well that can include either a greater or fewer total and/or sampled steps. Moreover, the ratio of sampled steps to the total number of steps can be increased and/or decreased as well and shall not be limited to the 20:5 ratio described in this exemplary embodiment.

After the step 304 of comparing, if the compared samples match within a first tolerance, the step 306 of comparing the segment with the first treatment can occur. In this step, if there a match within the given tolerance, the entire segment (including all the steps for a particular field) are compared to the entire treatment field for which a match occurred within the tolerance. As each of the fields of the given segment and given treatment field are compared, the step of 308 of generating output data can be performed. The step 308 of generating output data can include storing these data either on a local computer readable medium (e.g., as part of a local-based computer system and/or server) or remotely (such as, for example, on a remote server). Alternatively, only the remaining portion of the segment is compared with the remaining portion of the treatment field during the step 306 of comparing. In this example, the first portions already compared in the step 304 have been compared and, therefore, a duplicative comparison can be avoided for the first portion of the field.

Whether or not a “match” occurs depends on tolerances set and how those comparisons are performed within a given step of a treatment field. For simplicity, assume a treatment field has five steps, two of which are to be compared as the sample of the treatment field with the sample of the segment of the treatment plan data. In this example, assume step 1 requires the equipment to begin at a first three-dimensional coordinate on the patient's body, to deliver a given dose of radiation for 100 ms, while rotating at a given angle to a second three-dimensional coordinate.

The remaining four steps of this field are similar in this example, and vary in their coordinates, angles of rotation, duration of dose, at intensity of dose. As the equipment executes these steps, it can record each of these factors (e.g., start and stop coordinates of the step, duration of the dose, intensity of the dose, and rotational angles). Tolerances can be set to determine whether not a match occurs by imposing a +/−percentage for one or more of these factors. In other words, the steps performed by the equipment match within +/−5% of the steps set forth in the treatment plan data, a match will occur. Additionally, if multiple matches occur within a given set of comparisons, the match that will be outputted will be the one that has most recently been matched.

In one example, if the segment of the treatment plan data for a given step calls for a dose specification of 100 monitor units (MUs) and the treatment field records 95 MU, a match will occur because the actual dose was only 5% below what the treatment plan called for. Otherwise, the match will not occur and it will be assumed that the comparison is outside this tolerance because the treatment field being compared is a different field than the field of the segment in the treatment plan data. Although +/−5% tolerance is used in this example, a smaller or greater degree of variance between the treatment field and the segment can be applied equally as well.

Typically, this tolerance can be adjusted by the medical professional on a case-by-case basis based on the type of treatment, previous treatment data, etc. Furthermore, a “match” can occur even if fewer than all of these factors fall within a given tolerance. For example, if three of the factors (e.g., duration, dose, and angle) are within the given tolerance, but the remaining ones (e.g., start and end coordinates) are outside the tolerance, a match can still occur. In other words, the level of tolerances and what constitutes a “match” can be programmed and modified at a later time to meet the particular needs of a medical professional for a given treatment.

The step 308 of generating output data can include outputting data into a data file, data structure, or the like (such as, for example, a log file and/or other contiguous or non-contiguous data set). The step 308 of generating output data can occur in real-time as each step of a treatment field is compared with a step of the segment and, thus, stored directly on to a computer readable medium as the comparisons are completely within the given field. Alternatively, the step 308 of generating output data can include outputting the results of the comparison into a buffer. Once the comparison of a given filed is complete, the data stored in the buffer can be transmitted at a later time to a computer readable memory.

It is important to note that the step 308 of generating output data can similarly occur in real-time or otherwise during the step 304 of comparing a sample of a segment of treatment plan data. That is, as the first steps of a particular field in a given treatment field are being compared, the results of the comparison can be stored in a computer readable medium, or alternatively, in a buffer as described above. If the comparison results in a match within the tolerance, the data can be preserved and the step of 306 can be performed. In one example, the results of the comparison in step 306 can be appended with the results of the step 304 comparison. In another example (for example with use of a buffer), the comparison results of step 304 can be stored in a buffer and at a later time, the these results can be transferred to a computer readable memory so that the comparison results of the step 306 can be stored in the buffer.

The method 300 can further include the step 310 of comparing a sample of a subsequent segment of treatment plan data with a sample of a subsequent treatment field segment after completing the step 308 of generating an output. This comparison is described in greater detail below (the concept of a “subsequent” segment and/or treatment field is also described in greater detail below, with specific reference to FIG. 4). If the step 304 comparison does not result in a match within a first tolerance, the step 314 of comparing the sample of the segment with a sample of a subsequent treatment field can occur. In this step, the originally compared portion (e.g., sample) of the segment can be compared with a portion (e.g., sample) of another treatment field. For example, this can include the next treatment field (i.e., field at the next memory location) among the treatment fields in the treatment field data.

As similarly described with reference to the step 304 of comparing a sample of the first segment of treatment plan data with a sample of a first treatment field, if a match occurs within a first tolerance, the remaining portions of the subsequent treatment field can be compared with the remaining portions of the segment. This can occur during the step 316 of comparing the segment with the subsequent treatment field. Just as described above in connection with the step 306 of comparing the segment with the first treatment field, either the entire subsequent treatment field can be compared with the segment or just the remaining portions of the subsequent treatment field can be compared with the remaining portions of the segment at the step 316.

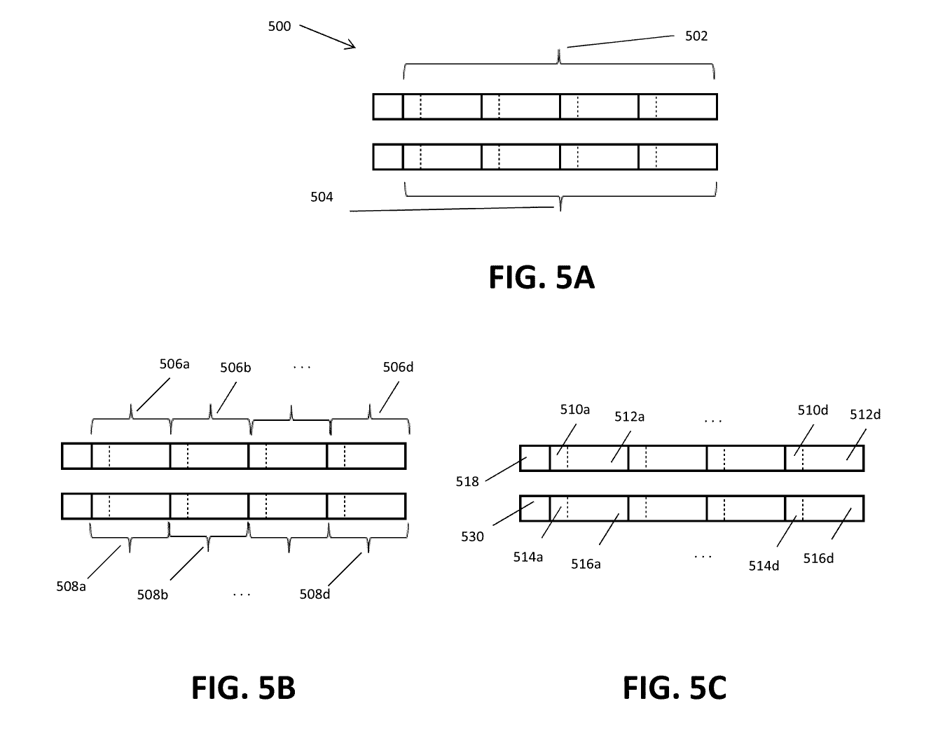

If the comparison in step 314 does not result in a match within a first tolerance, the step 318 of comparing the sample of the segment with a sample of another subsequent treatment field can occur. In other words, if a match does not occur with the compared samples, the method 300 can continue comparing subsequent treatment fields until a match occurs. This process is illustrated in greater detail, for example, in FIG. 5D.

Furthermore, the step 320 of generating output data comprising the results of the step of comparing the sample of the segment and the sample of a subsequent treatment field if a match occurs within the first tolerance can be performed. This step can be performed in a manner similar to the step 308 of generating output data as described above and thus, in the interest of clarity and brevity, further discussion of how the data are outputted and stored (e.g., on a computer readable medium, buffer, or the like) will be omitted.

Finally, the step 312 of analyzing the output data to assess the level of quality assurance of the medical treatment can be performed. In this step, the results of the comparison can be analyzed to determine, for example, how closely the medical treatment performed for a particular patient compared to the intended treatment contained in the treatment plan data. This analysis can be performed on the data set as a whole, on a field-by-field basis, or on a step-by-step basis within each of the discrete fields in the patient's treatment plan.

By performing these comparisons, additional data, such as data structures or the like, or representations of data, such as charts, graphs, etc. can be generated to determine the dosages delivered during the treatment. These data can then be used to resolve any discrepancies that may have occurred during throughout a particular field or fields, or can provide medical professionals with information to help modify treatment plans in the future. In other words, these data provide quality assurance levels of the 3D dosage applied during a particular treatment and verify whether or not the treatment was successful and/or to what degree it was successful.

FIG. 4 illustrates a flow diagram depicting a second embodiment of exemplary steps for carrying out a method for verifying the accuracy of medical treatment. The method 400 can include the step 402 of receiving medical treatment data including one or more treatment fields and the step 404 of comparing a first portion of a current segment of treatment plan data with a first portion of a current treatment field. For example, the step 402 of receiving medical treatment data can include receiving either raw data, or data in a structured form (e.g., a data structure such as, for example, a list, table, log, or the like). These medical treatment data can be supplied from the equipment performing the treatment, or alternatively, provided from a separate system, computer, server, or the like. The equipment performing the treatment can include a linear accelerator or other equipment for performing radiation-type treatment on a patient.

The treatment fields (e.g., 206 as illustrated in FIG. 2) can represent a series of steps to be performed for the treatment of a patient. For example, a field can include a set of steps to instruct the equipment to direct energy, such as a linear accelerator beam, through a series of motions based on three-dimensional coordinates, rotational angles, and the like. Further, a treatment field can instruct the equipment to direct a beam at a portion of a patient to perform medical treatment. During a patient's therapy treatment, the equipment can execute steps through one or more of these discrete fields by turning the beam on and off at particular locations and throughout a series of rotations to perform the particular treatment required in accordance with a patient's treatment plan.

As noted above, the initial comparison of portions of segments to portions treatment fields can begin with the comparison of a “current” segment of treatment plan data with a first portion of a “current” treatment field (e.g., step 404). The “current” segment and “current” treatment field are those segments and fields, respectively, that are currently being compared. For example, for the initial comparison of treatment plan data with medical treatment data, the current segment and the current treatment field can include the first segment and the first treatment field stored among the treatment plan data set and medical treatment data set, respectively.

In other examples, the current segment and current treatment field can be a segment and treatment field, respectively, other than the first. However, regardless of which of the segments and treatment fields are set as the “current” segment and treatment field initially, the current segment and field will always be the one that is currently being examined and/or analyzed, such as through a comparison. Therefore, the “current” segment and “current” treatment field differ from the “subsequent” segment and “subsequent” treatment field in that the “subsequent” segment and treatment field, are the segment and treatment field, respectively, that are to be examined and/or analyzed (e.g., compared) after the current segment and treatment field are examined and/or analyzed.

The “current” and “subsequent” segments and treatment fields can be tracked through the use of memory pointers, look-up tables, or other programming techniques for storing, tracking, and analyzing memory locations. For example (using the memory pointer in a non-limiting illustrative embodiment), a “current” segment can be initially set to the first memory location of the first segment stored among all the treatment plan data. Keeping with this example, the subsequent segment can be set to the first memory location of the second segment stored among all the treatment plan data.

When the method 400 sets the subsequent segment as the current segment (for example, as in step 410 as described in greater detail below), the initial current segment pointer can be pointed to the memory location presently being pointed to by the subsequent segment pointer. In turn, the initial subsequent segment pointer can be pointed to another segment among the treatment plan data (e.g., the third segment of the treatment plan data). The current and subsequent treatment field can be similarly updated. The process of setting and resetting the current and subsequent segments and current and subsequent treatment fields can continue throughout each segment and treatment field of the treatment plan data and medical treatment data, respectively.

The locations of current and subsequent segments and treatment fields (e.g., memory locations) can be stored along each of their respective segments and fields, or in the alternative, can be stored in a separate memory device (not shown). Additionally, as mentioned above, a separated look-up table can be used in additional to, or in lieu of, the storage of the memory locations such that when a subsequent segment or subsequent treatment field becomes a current segment or current treatment field, the memory locations can be resolved by referring to the table.

The step 404 of comparing a first portion of a segment of treatment plan data with a first portion of a first treatment field can be performed by comparing a portion of the first few steps/instructions in a given segment with first few steps/instructions of a given treatment field. In an exemplary and non-limiting illustrative embodiment, a field may include fifteen discrete sets of coordinates and/or angles for which the equipment must navigate to compete a particular fields. In one example, the step 404 can include comparing a first portion of the treatment field and segment of the treatment plan, such as the first three of these fifteen steps.

Although this example employs fifteen total steps, the first three of which are being compared initially, other examples are contemplated as well that can include either greater or fewer total and/or sampled steps. Moreover, the ratio of sampled steps to the total number of steps can be increased and/or decreased as well and shall not be limited to the 15:3 ratio described in this exemplary embodiment.

After the step 404 of comparing, if the compared portions match within a first tolerance, the step 406 of comparing the current segment with the current treatment can occur. In this step, if there is a match within the given tolerance, the entire segment (including all the steps for a particular field) are compared to the entire treatment field for which a match occurred within the tolerance. As each of the fields of the given segment and given treatment field are compared, the step of 308 of generating output data can be performed. Alternatively, only the remaining portion of the segment is compared with the remaining portion of the treatment field during the step 406 of comparing. In this example, the first portions already compared in the step 404 have been compared and, therefore, a duplicative comparison can be avoided for the first portion of the field.

Whether or not a “match” occurs depends on tolerances set and how those comparisons are performed within a given step of treatment field. For simplicity, assume a treatment field has five steps, two of which are to be compared as the portion of the treatment field with the portion of the segment of the treatment plan data. In this example, assume step 1 requires the equipment to begin a first three-dimensional coordinate on the patient's body, to deliver a given dose of radiation for 50 MU while rotating at a given angle to a second three-dimensional coordinate.

The remaining steps of this field are similar in this example, and vary in their coordinates, angles of rotation, duration of dose, at intensity of dose. As the equipment executes these steps, it can record each of these factors (e.g., start and stop coordinates of the step, duration of the dose, intensity of the dose, and rotational angle). Tolerances can be set to determine whether not a match occurs by imposing a +/−percentage (or +/−distance, +/−angle, etc.) for one or more of these factors. In other words, the steps performed by the equipment match within +/−2% of the steps set forth in the treatment plan data, a match will occur.

The step 408 of generating output data can include outputting data into a data file, data structure, or the like (such as, for example, a log file and/or other contiguous or non-contiguous data set). The step 408 of generating output data can be similarly performed as described in conjunction with step 308 of FIG. 3 and, therefore, will not repeated here in the interest of clarity and brevity.

The method 400 can further include the step 410 of comparing the first portion of the current segment if the step 406 of comparing the current segment does not result in a match within the first tolerance. In other words, because the previously compared portion of the current treatment field did not match within a tolerance of the initial, current segment, another segment is compared (here, a subsequent segment) in order to determine if a match occurs. From there, the step 412 of setting the subsequent treatment field as the current treatment field can occur and the step 404 of comparing, the step 406 of comparing, and the step 408 of comparing can be repeated such that a comparison of this “new” current segment (i.e., previous subsequent segment) with the current treatment field is performed to determine if there is a match. This process may be repeated until a match is found of the current treatment field among the segments of the treatment plan data.

Additionally, the step 414 of setting a subsequent segment as the current segment and setting a subsequent treatment field as the current treatment field in response to the generating output data can occur. In other words, if a “match” occurred based on the step 406 of comparing, the current segment and treatment field are a match and, thus, the next treatment field can be compared with next segment and so on. Once the subsequent segment is set as the current segment, the step 404 of comparing, the step 406 of comparing, and the step 408 of comparing can be repeated (e.g., step 416) such that by comparing this “new” current segment (i.e., previous subsequent segment) with the “new” treatment field to determine if there is a match. This process may be repeated until a match is found of the current treatment field among the segments of the treatment plan data. The recursive process can be further illustrated by the exemplary comparisons as set forth in FIG. 5D.

Finally, the method 400 can further include the step 418 of setting a flag associated with the current treatment field in response to the step 408 of generating output data. In this step, the flag can include a bit, semaphore, or the like for signaling when a treatment field and/or segment matched. By setting this flag, the method 400 can subsequently skip over any segments and/or treatment fields that have been previously flagged (i.e., because of a match) in order to ensure that segments and fields already known to match are not re-compared, thus increasing the overall speed and efficiency of the comparison of all segments and treatment fields among the medical treatment data and treatment plan data. The flags can be stored within each of their respective treatment fields and/or segments (for example, as illustrated in FIG. 5C as treatment field flag 518 and segment flag 520), or in the alternative, can be stored in a separate memory locations.

FIG. 5A illustrates a second embodiment of medical data in accordance with the present invention. FIG. 5B illustrates the second embodiment of medical data as depicted in FIG. 5A illustrating certain features in accordance with the present invention. FIG. 5C illustrates the second embodiment of medical data as depicted in FIG. 5A illustrating additional features in accordance with the present invention. FIG. 5D illustrates a second embodiment of exemplary steps for verifying the accuracy of medical treatment in accordance with the second embodiment of medical data as depicted in FIG. 5A. These Figures will described in conjunction with one another.

With specific reference to FIGS. 5A-5C, many of the features of data structure 500 can be similarly embodied as described in conjunction with data structure 200 of FIG. 2. That is, data structure 500 can include medical treatment data 502 and treatment plan data 504. The medical treatment data 502 can include one or more treatment fields (e.g., 506a-506d), each of which can include first and second portions/samples, respectively (e.g., 510a and 512b). Similarly, treatment plan data 504 can include one or more segments (e.g., 508a-508d), each of which can include first and second portions/samples, respectively (e.g., 514a and 516b). Because these elements are similarly described in conjunction with FIG. 2, the examples and embodiments used to describe these particular elements with reference to FIG. 2 can similarly be used to illustrate the corresponding features of FIGS. 5A-5D. Accordingly, additional disclosure regarding these elements will not be repeated here for clarity and brevity.

Additionally, one or more flags (e.g., treatment field flag 518 and segment flag 520) can be associated with the medical treatment data 502 and treatment plan data 504, respectively. These flags can include a bit, semaphore, or the like for signaling when a treatment field and segment matched. For example, if segment 508a “matches” treatment field 506a, neither segment 508a nor treatment field 506a need to be compared with another treatment field or segment, respectively. In order to prevent duplicative comparison steps, therefore, both the treatment field flag 518 and the segment flag 520 can be set for treatment field 506a and segment 508a, respectively so that these are no longer compared again (see, e.g., step (d) of FIG. 5D as described in greater detail below).

Alternatively, one or neither of the flags 518, 520 can be set even if a match occurs. For example, if a particular segment contains steps that must be performed over multiple fields throughout a treatment, the segment can be compared to multiple fields even after a match between the given segment and field occurs. For example, assume the steps performed in field 506a must be performed across multiple fields (e.g., field 506a and 506d) and that those steps are represented in segment 508a. In this example, the flag 518 for field 506a can be set after it matches with segment 508a, but the flag 520 associated with segment 508a would not be set because it can be used to be compared at a later time with segment 508d.

As illustrated in FIG. 5C, flags 518 and 520 can be stored at a single location respective to each of the medical treatment data 502 and the treatment plan data 504. Alternatively (although not shown in the figures), an individual flag can be stored in, and associated with, each of the respective treatment fields 506 and segments 508. In this example each of treatment field and segment flags (518 and 520, respectively) can be analyzed for each comparison, before the comparisons take place, so that a set flag can trigger to the system to move to a subsequent field and/or segment. In other words, by setting this flag, the system subsequently skip over any segments and/or treatment fields that have been previously flagged (i.e., because of a match) in order to ensure that segments and fields already known to match are not re-compared, thus increasing the overall speed and efficiency of the comparison of all segments and treatment fields among the medical treatment data and treatment plan data.

Referring specifically, to FIG. 5D, a particular comparison is illustrated at a step-by-step basis on the data structure 500 as illustrated in FIGS. 5A-5C. Although this data structure illustrates four treatment fields 506, four segments 508, and a single flag field (adapted to store at least one flag for each of the respective fields and/or segments), other structures are contemplated as well and should not be limited to this particular example.

Beginning with step (a), the current treatment field and current segment are both initially set to the first treatment field (e.g., FIG. 5B, 506a) and first segment (e.g., FIG. 5B, 508a) respectively. Once set, the first portion of the first segment is compared to the first portion of the first treatment field. In this example, a match is determined (i.e., within a first tolerance) and, thus, the process continues with step (b). Step (b) compares the second portion of the first segment with the second portion of the first treatment field and outputs the data (as shown in step (c), for example). The data at this step can be stored in a log file, raw data file, data structure, etc.

After the match occurs, a flag bit can be set for both the first segment and the first treatment field (e.g., flags 518 and 520 of FIG. 5C). This is represented, for example, in step (d) as the shaded area. Because the flags are set, the first segment and the first treatment field are no longer subject to comparison for the given medical treatment data and treatment plan data in this example. Because a match occurred at step (b), the subsequent treatment field (in this example, the subsequent treatment field is the next (e.g., second) treatment field) is set as the current treatment field and the subsequent segment (in this example, the subsequent segment is the next (e.g., second) segment) is set as the current segment. This is represented in this example as step (d) where now a first portion of the newly set current segment (e.g., second segment) can be compared with a first portion of the newly set treatment field. In this example, a match does not occur.

Because a match did not occur, the current segment remains the same but the subsequent treatment field (e.g., third treatment field) is set as the current treatment field so that it can be compared with the current segment (i.e., second segment). This is represented as step (e). Again, in this example, no match occurs and, thus, no output is read to the output data (for example, the data shown at step (c)). This is represented as step (f).

Again, because a match did not occur, the current segment remains the same but the subsequent treatment field (e.g., fourth treatment field) is set as the current treatment field so that it can be compared with the current segment (i.e., second segment). This is represented as step (g). In this comparison, a match is determined (i.e., within a first tolerance) and, thus, the process continues with step (h). Step (h) compares the second portion of the second segment with the second portion of the fourth treatment field and outputs the data (as shown in step (i), for example). The data at this step can be stored in a log file, raw data file, data structure, etc. The outputted data can be appended to the first outputted data as shown in step (c) or, in the alternative, stored separately in a computer readable medium.

After this match occurs, a flag bit can now be set for both the second segment and the fourth treatment field (e.g., flags 518 and 520 of FIG. 5C). This is represented, for example, in step (j) as the shaded area. Because the flags are set, the first and second segments and the first and fourth treatment fields are no longer subject to comparison for the given medical treatment data and treatment plan data in this example. Because a match occurred at step (h), the subsequent treatment field (e.g., second) is set as the current treatment field and the subsequent segment (in this example, the subsequent segment is the next (e.g., third) segment) is set as the current segment. This is represented in this example as step (j) where now a first portion of the newly set current segment (e.g., third segment) can be compared with a first portion of the newly set treatment field. In this example, a match occurs and, thus, second portion of the third segment is compared with the second portion of the second treatment field and the result of the comparison is outputted as shown in step (I).

After this match occurs, a flag bit can now be set for both the third segment and the second treatment field (e.g., flags 518 and 520 of FIG. 5C). This is represented, for example, in step (m) as the shaded area. Because the flags are set, the first, second, and third segments and the first, second, and fourth treatment fields are no longer subject to comparison for the given medical treatment data and treatment plan data in this example. Because a match occurred at step (k), the subsequent treatment field (e.g., third) is set as the current treatment field and the subsequent segment (e.g., fourth) is set as the current segment. This is represented in this example as step (m) where now a first portion of the newly set current segment (e.g., fourth segment) can be compared with a first portion of the newly set treatment field. In this example, a match occurs and, thus, second portion of the fourth segment is compared with the second portion of the third treatment field and the result of the comparison is outputted as shown in step (o).

Although this particular examples proceeded in a linear fashion starting with the first segment and first treatment field, and progressing such that the subsequent segment and subsequent treatment field are compared as the second segment and field, and so on, other examples can include one or more of the subsequent segments and fields being assigned to segments and fields other than those that are located in the adjacent and/or nearest memory locations to the current segments and treatment fields. In other words, the process described in these examples can be designed to skip back and forth among memory locations if needed and are not so limited by the progression described in this particular example.

Moreover, in an alternative embodiment, first and second portions/samples can be omitted such that each treatment field and each segment is limited to only one portion each. In this example, the steps requiring a comparison of a subset of a segment with a subset of a treatment field may be omitted. In lieu of this sampling of a subset, the entire segment can be compared with the entire treatment field to determine if a match occurs within a given tolerance. For higher rates of matches, this can improve the overall efficiency of the comparisons because it eliminates the step of an initial comparison before comparing either the remaining portion/s of the segments and treatment fields or entire segments and treatment fields. However, with higher miss rates (i.e., no match occurring), the efficiency can reduced in comparison with the dual-step comparison process described, for example, in conjunction with FIGS. 3 and 4. Accordingly, programmers and/or medical professionals can have the ability to adjust with methodology to choose on—a case-by-case basis—to maximize the efficiency of these comparisons.

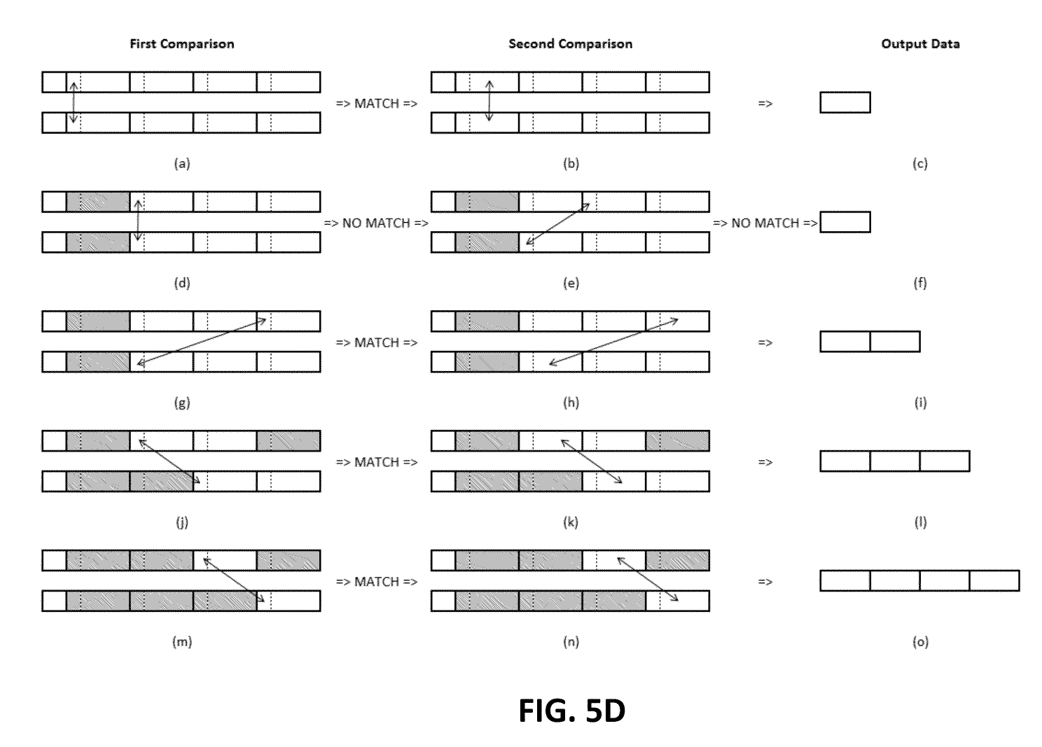

FIG. 6 illustrates an embodiment of a computer readable medium configured to store an application for verifying the accuracy of medical treatment in accordance with certain aspects of the inventions described herein. Apparatus 600 can include a computer readable medium 604 can include any medium that that can be used in conjunction with the computer readable instructions, programs, or applications, such as, for example, the applications and/or programs described in conjunction with the process steps described in greater detail herein. For example, computer readable medium 604 can be configured to store a program 602 for verifying the accuracy of medical treatment in accordance with a treatment plan. The program 602 is adapted to execute instruction for performing various steps. For example, in an exemplary and non-limiting illustrative embodiment, the steps of the method 300 and/or method 400 as described above in conjunction with FIGS. 3 and 4, respectively.

Program 602 can include programs, instructions, firmware, software, hardware, or any combination thereof for instructing a computer or other electronic device for performing and/or carrying out a series of steps and/or instructions in accordance with the process steps described above (such as, for example, FIGS. 3 and 4). The computer readable instructions can include any code and/or instruction that is adapted to be read by a computer, such as, assembly, machine, executable, non-executable, compiled, or uncompiled code, or any other instructions adapted to be read by a computer or electric device with an arithmetic logic unit or the like.

In an exemplary and non-limiting illustrative embodiment, the computer readable medium 604 can include a computer readable storage medium (“CRSM”). The computer readable storage medium can take many forms, including, but not limited to, non-volatile media and volatile media, floppy disks, flexible disks, hard disks, magnetic tape, other magnetic media, CD-ROMs, DVDs, or any other optical storage medium. Computer readable storage media can further include RAM, PROM, EPROM, EEPROM, FLASH, combinations thereof (e.g., PROM EPROM), or any other memory chip or cartridge.

The computer readable medium 604 can further include computer readable transmission media (“CRTM”). These transmission media can include coaxial cables, copper wire and fiber optics. Transmission media may also take the form of acoustic or light waves, such as those generated during radio frequency, infrared, wireless, or other media comprising electric, magnetic, or electromagnetic waves.

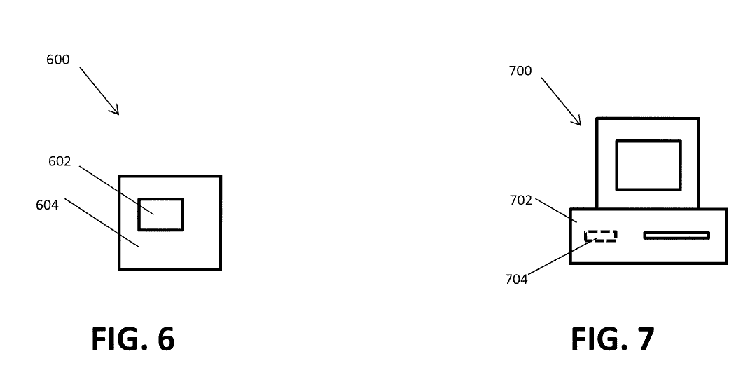

FIG. 7 illustrates an embodiment of a system for verifying the accuracy of medical treatment in accordance with certain aspects of the inventions described herein. It is important to note that several features described with reference to FIG. 6 are similarly illustrated in FIG. 7 (e.g., computer readable medium 604 in FIG. 6 and computer readable medium 704 in FIG. 7). As such, the computer readable medium 704 can be similarly described by the examples and embodiments for the computer readable medium 604 of FIG. 6.

System 700 can include a computer readable medium 704, and computer 702. The computer readable medium 704 can include a program (not shown) which, when executed, can perform various steps (e.g., the method 300 and method 400 as illustrated in FIGS. 3 and 4, respectively). In one example, computer readable medium 704 can include a storage medium, such as a hard disk drive or FLASH memory drive. In another example, computer readable medium 704 can be located distally and/or remotely from computer 702 (e.g., on a server) such that the data, media, and/or instructions stored on it can be transmitted to computer 702.

Computer 702 can include any laptop, netbook, notebook, desktop computer, or any other computer system that can be employed for outputting and analyzing data. In other words, the data outputted from the various output steps described above (e.g., in conjunction with FIGS. 3 and 4) can be sent to computer 702 to perform the analysis steps (e.g., for example, step 312 of analyzing the output data to assess quality as illustrated in FIG. 3).

Although not explicitly recited throughout the description related to the process steps set forth in FIGS. 3 and 4, certain aspects of the inventions that are described in conjunction with the apparatuses and systems above (such as, for example, a particular function of element) can be carried out as one or more process steps and/or instructions adapted to executed those one or more process steps. For example, medical treatment data (e.g., 202 as shown in FIG. 2) can be stored as a log on computer readable memory 704. In this example, computer 702 can continually monitor computer readable memory 704 to detect if additional medical treatment data (e.g., new field of a particular patient's log) is added. If so, the steps described in FIGS. 3 and 4 can begin to process the newly identified field data.

The figures described above and the written description of specific structures and functions below are not presented to limit the scope of what Applicants have invented or the scope of the appended claims. Rather, the figures and written description are provided to teach any person skilled in the art to make and use the inventions for which patent protection is sought. Those skilled in the art will appreciate that not all features of a commercial embodiment of the inventions are described or shown for the sake of clarity and understanding. Persons of skill in this art will also appreciate that the development of an actual commercial embodiment incorporating aspects of the present inventions will require numerous implementation-specific decisions to achieve the developer's ultimate goal for the commercial embodiment.

Such implementation-specific decisions may include, and likely are not limited to, compliance with system-related, business-related, government-related, and other constraints, which may vary by specific implementation, location and from time to time. While a developer's efforts might be complex and time-consuming in an absolute sense, such efforts would be, nevertheless, a routine undertaking for those of skill in this art having benefit of this disclosure. It must be understood that the inventions disclosed and taught herein are susceptible to numerous and various modifications and alternative forms. Lastly, the use of a singular term, such as, but not limited to, “a,” is not intended as limiting of the number of items. Also, the use of relational terms, such as, but not limited to, “top,” “bottom,” “left,” “right,” “upper,” “lower,” “down,” “up,” “side,” and the like are used in the written description for clarity in specific reference to the figures and are not intended to limit the scope of the invention or the appended claims.

Particular embodiments of the invention may be described below with reference to block diagrams and/or operational illustrations of methods. It will be understood that each block of the block diagrams and/or operational illustrations, and combinations of blocks in the block diagrams and/or operational illustrations, can be implemented by analog and/or digital hardware, and/or computer program instructions. Such computer program instructions may be provided to a processor of a general-computer, special purpose computer, ASIC, and/or other programmable data processing system. The executed instructions may create structures and functions for implementing the actions specified in the block diagrams and/or operational illustrations. In some alternate implementations, the functions/actions/structures noted in the figures may occur out of the order noted in the block diagrams and/or operational illustrations. For example, two operations shown as occurring in succession, in fact, may be executed substantially concurrently or the operations may be executed in the reverse order, depending upon the functionality/acts/structure involved.

Computer programs for use with or by the embodiments disclosed herein may be written in an object oriented programming language, conventional procedural programming language, or lower-level code, such as assembly language and/or microcode. The program may be executed entirely on a single processor and/or across multiple processors, as a stand-alone software package or as part of another software package.

Other and further embodiments utilizing one or more aspects of the inventions described above can be devised without departing from the spirit of Applicant's invention. It should be appreciated by those of skill in the art that the techniques disclosed in the disclosed embodiments represent techniques discovered by the inventor(s) to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the scope of the invention. Other variations of the systems, apparatuses, and methods can be included in combination with each other to produce variations of the disclosed embodiments. Discussion of singular elements can include plural elements and vice-versa.

In some alternate implementations, the functions/actions/structures noted in the figures can occur out of the order noted in the block diagrams and/or operational illustrations. For example, two operations shown as occurring in succession, in fact, can be executed substantially concurrently or the operations can be executed in the reverse order, depending upon the functionality/acts/structure involved.

The order of steps can occur in a variety of sequences unless otherwise specifically limited. The various steps described herein can be combined with other steps, interlineated with the stated steps, and/or split into multiple steps. Similarly, elements have been described functionally and can be embodied as separate components or can be combined into components having multiple functions.

The inventions have been described in the context of preferred and other embodiments and not every embodiment of the invention has been described. Obvious modifications and alterations to the described embodiments are available to those of ordinary skill in the art. The disclosed and undisclosed embodiments are not intended to limit or restrict the scope or applicability of the invention conceived of by the Applicants, but rather, in conformity with the patent laws, Applicant intends to fully protect all such modifications and improvements that come within the scope or range of equivalent of the following claims.

What is claimed is:

1. A method for verifying the accuracy of medical treatment in accordance with a treatment plan, wherein the method comprising the following steps:
receiving medical treatment data comprising one or more treatment fields;
comparing a sample of a segment of treatment plan data with a sample of a first treatment field;
comparing the segment with the first treatment field if the compared samples match within a first tolerance; and
generating output data comprising the results of the step of comparing the segment with the first treatment field if a match occurs within the first tolerance.

2. The method according to claim 1 further comprising the step of comparing the sample of the segment with a sample of a subsequent treatment field if the comparison of the segment with the first treatment field did not result in a match within the first tolerance.

3. The method according to claim 2 further comprising the step of comparing the segment with the subsequent treatment field if the compared samples match within a first tolerance.

4. The method according to claim 3 further comprising the step of generating output data comprising the results of the step of comparing the sample of the segment and the sample of a subsequent treatment field if a match occurs within the first tolerance.

5. The method according to claim 2 further comprising the step of comparing the sample of the segment with a sample of another subsequent treatment field if the compared samples do not match within a first tolerance.

6. The method according to claim 1 further comprising the step of comparing a sample of a subsequent segment of treatment plan data with a sample of a subsequent treatment field segment after completing the step of generating output data.

7. The method according to claim 1 further comprising the step of analyzing the output data to assess the level of quality assurance of the medical treatment.

8. A computer readable storage medium configured to store a program for verifying the accuracy of medical treatment in accordance with a treatment plan, wherein the program is adapted to execute instructions for performing the following steps, comprising:
receiving medical treatment data comprising one or more treatment fields;
comparing a sample of a segment of treatment plan data with a sample of a first treatment field;
comparing the segment with the first treatment field if the compared samples match within a first tolerance; and
generating output data comprising the results of the step of comparing the segment with the first treatment field if a match occurs within the first tolerance.

9. The computer readable storage medium according to claim 8 further comprising the step of comparing the sample of the segment with a sample of a subsequent treatment field if the comparison of the segment with the first treatment field did not result in a match within the first tolerance.

10. The computer readable storage medium according to claim 9 further comprising the step of comparing the segment with the subsequent treatment field if the compared samples match within a first tolerance.

11. The computer readable storage medium according to claim 10 further comprising the step of generating output data comprising the results of the step of comparing the sample of the segment and the sample of a subsequent treatment field if a match occurs within the first tolerance.

12. The computer readable storage medium according to claim 9 further comprising the step of comparing the sample of the segment with a sample of a subsequent treatment field if the comparison of the segment with the first treatment field did not result in a match within the first tolerance if the compared samples do not match within a first tolerance.

13. The computer readable storage medium according to claim 8 further comprising the step of comparing a sample of a subsequent segment of treatment plan data with a sample of a subsequent treatment field segment after completing the step of generating output data.

14. The computer readable storage medium according to claim 8 further comprising the step of analyzing the output data to assess the level of quality assurance of the medical treatment.

15. A system for verifying the accuracy of medical treatment in accordance with a treatment plan, wherein the system comprises:
a computer; and
a computer readable storage medium configured to store a program, wherein the program is adapted to execute instructions for performing the following steps, comprising:
receiving medical treatment data associated with a first patient, wherein the medical treatment data includes a plurality of treatment fields;
comparing a first portion of a current segment of treatment plan data with a first portion of a current treatment field of the medical treatment data;
comparing the current segment with the current treatment field if the first portion of the current segment matches the first portion of the current treatment field within a first tolerance; and
generating output data comprising the results of the comparing the current segment with the current treatment field step in response to the step of comparing the current segment.

16. The system according to claim 15 further comprising the step of comparing the first portion of the current segment to a first portion of a subsequent treatment field if the step of comparing the current segment does not result in a match within the first tolerance.

17. The system according to claim 16 further comprising setting the subsequent treatment field as the current treatment field and repeating the step of comparing the current segment with the current treatment field and the step of generating output data.

18. The system according to claim 15 further comprising setting a subsequent segment as the current segment and setting a subsequent treatment field as the current treatment field in response to the step of generating output data.

19. The system according to claim 18 further comprising repeating the comparing and generating steps according to claim 15.

20. The system according to claim 15 further comprising setting a flag associated with the current treatment field in response to the step of generating output data.
